(19) United States
(12) Patent Application Publication
(10) Pub. No.: US 2002/0147480 A1
(43) Pub. Date: Oct. 10, 2002

(54) Treatment of lipid pool

(76) Inventor: Donald S. Mamayek, Mountain View, CA (US)

Correspondence Address: LYON & LYON LLP, 633 WEST FIFTH STREET, SUITE 4700, LOS ANGELES, CA, 90071, US

(21) Appl. No.: 09/826,762

(22) Filed: Apr. 4, 2001

(51) Int. Cl. A61F 7/00

(52) U.S. Cl. 607/105

(57) ABSTRACT

A method and apparatus for solidifying inflamed and unstable plaque on the interior of a blood vessel including inserting a catheter assembly into a human blood vessel while the assembly is in a first, collapsed configuration, advancing the catheter assembly through the blood vessel, stopping at predetermined intervals and expanding the catheter assembly to a second, expanded configuration such that a plurality of temperature detectors contact the interior wall of the blood vessel. The areas of increased temperature are indicative of inflamed and unstable plaque, which is to be stabilized using at least one multi-temperature device, such as a Peltier device, to solidify the plaque and reduce the possibility of a myocardial infarction.

### FIELD OF THE INVENTION

The present invention relates, in general, to methods and apparatuses for solidifying inflamed/unstable plaque on the interior of a blood vessel.

### DISCUSSION OF RELATED ART

Arterial plaque of varying size and location can develop in a human cardiovascular system. A build-up of plaque over a number of years can cause blood vessel occlusion and associated heart failure. Lipid pools are trapped reservoirs of potentially dangerous plaque material that, when released into the blood stream, can cause complete stoppage or major reduction or disruption of blood flow in a vessel. If this occurs in a cardiovascular vessel or other critical vessel, it can cause sudden flow stoppage and possibly death. Lipid pools containing arterial plaque that becomes inflamed and unstable incur an increased risk of ulceration and rupture. When a lipid pool rupture develops, the material flows out into the vessel, causing the blood to quickly thicken or coagulate and form a blood clot. This can cause a myocardial infarction.

Importantly, lipid pools containing inflamed and unstable plaque are indicated by associated localized temperature variations of the interior blood vessel wall surface. Specialized temperature sensors such as those disclosed in U.S. Pat. No. 5,871,449 can be used to sense localized temperature variations of blood vessel wall surfaces within human arteries. These sensors can detect the presence of lipid pools containing inflamed and unstable plaque, which have up to a two and a half degree increase in artery wall temperature.

Various methods of plaque removal include compression or removal of the plaque, which typically results in residual sites of injury and a predisposition toward recurrent plaque occlusion. These methods are alternatives to the more traumatic and expensive coronary bypass procedures. One example of a plaque removal method involves the use of an atherectomy device for physically cutting and removing the plaque from the affected arteries. Another commonly used method to increase blood flow involves balloon angioplasty, which reduces the arterial blockage by dilation of the lumen of the artery. Before the balloon is inserted, a laser may be used to create a channel in the artery by heating and melting the plaque. Unfortunately, these methods suffer from some serious drawbacks.

Regarding atherectomy and angioplasty methods, the devices used to remove the plaque often cause damage to the interior walls of the artery due to scarring caused by cutting and misdirection of laser energy. Moreover, laser energy can burn a hole through the wall of an artery if the laser is not controlled properly. In addition, cutting or melting of liquid lipid pools can release toxic fluids into the blood stream and cause instantaneous blood clots. Accordingly, there exists a need to develop a safer method of removing unstable plaque from the interior of an artery.

The present invention involves the use of multi-temperature devices or heating-cooling devices, such as Peltier devices, to apply heat and/or cold to solidify a lipid pool such that the lipid pool is stabilized and less likely to create a sudden release of plaque. The multi-temperature devices can be used alone or in combination with the aforementioned or other techniques for removing the solidified plaque.

### SUMMARY OF THE INVENTION

A separate aspect of the invention involves a method and apparatus for solidifying inflamed and unstable plaque on the interior wall of a blood vessel. The method initially entails inserting a catheter assembly into a human blood vessel while the assembly is in a first, collapsed configuration. Next, the catheter assembly is advanced through the blood vessel, stopped at predetermined intervals and expanded to a second, expanded configuration such that at least one temperature detector contacts the interior wall of the blood vessel. A noted increase in temperature of an area of the internal wall of the blood vessel is indicative of inflamed, unstable plaque, which may be stabilized by applying heat and/or cold to the areas of increased temperature to solidify the plaque and reduce the possibility of a sudden release into the bloodstream.

Another separate aspect of the invention involves a method and apparatus for eliminating inflamed and unstable plaque from the interior of a blood vessel by applying heat or cold using at least one multi-temperature device having a hot side and a cold side powered by an electrical current having a polarity. The multi-temperature device can be used to heat and or cool the lipid pool. An example of a multi-temperature device is a Peltier device, which has a cold side and a hot side. Other methods of coagulating or solidifying the lipid material use electrically generated resistance heating or pipes a heated gas or liquid media to the desired site. The cold temperature can also be generated by the expansion of liquid to a gas or by the injection of a cold liquid/gas media into a multi-temperature therapy plate. By reversing the polarity of the electrical current applied to a Peltier device, one can change the hot side to the cold side and vice-versa. In addition, the catheter assembly includes a therapy plate positioned in between the at least one multi-temperature device and the interior of the blood vessel wall that includes imbedded temperature sensors for sensing variation in blood vessel temperature at a plurality of areas along the interior wall of the blood vessel.

A further separate aspect of the invention involves a method and apparatus for removing undesired energy caused by at least one multi-temperature device by the process of heat sinking using a heat exchanger. The process of heat sinking includes circulating a liquid or gas through the heat exchanger located within the blood vessel to remove the excess energy from the procedural site. The heat exchanger may be lined with internal starburst fins or may include a large lumen to allow the passage of a probe, like a guide wire or catheter. The liquid can be condensed gas or refrigerant, water, or blood from the blood stream and the gas may be air. Air or gas must be contained within plumbing and not allowed to mix with the patient's blood.

Another separate aspect of the present invention involves a method and apparatus for eliminating inflamed and unstable plaque from the interior of a blood vessel by applying heat and/or cold using at least one multi-temperature device to solidify the plaque and then removing the solidified plaque resultant from the procedure using atherectomy.

An additional separate aspect of the invention involves a method and apparatus for eliminating inflamed and unstable plaque from the interior of a blood vessel using a catheter assembly including an expander, which may be a balloon, a plurality of multi-temperature devices, a therapy plate, temperature sensors, a temperature controller and a control box. The plurality of multi-temperature devices is stacked one on top of another to increase the overall thermal energy differential. The control box contains the circuitry to control temperature and to monitor the temperatures of the therapy plate and the vessel wall.

The invention may include any one of these separate aspects individually, or any combination of these separate aspects.

Other features and advantages of the invention will be evident from reading the following detailed description, which is intended to illustrate, but not limit, the invention.

### BRIEF DESCRIPTION OF THE DRAWINGS

The drawings illustrate the design and utility of preferred embodiments of the present invention, in which similar elements are referred to by common reference numerals.

FIG. 1 is a front plan view of an apparatus for treatment of lipid pools including a handle and a catheter assembly.

FIG. 2 is a cross-sectional view of the catheter assembly shown in FIG. 1 in an expanded configuration within a human artery.

FIG. 3A is a cross-sectional view of a first catheter assembly embodiment.

FIG. 3B is a cross-sectional view of second catheter assembly embodiment.

FIG. 3C is a cross-sectional view of third catheter assembly embodiment.

FIG. 3D is a cross-sectional view of fourth catheter assembly embodiment.

FIG. 4 is a plan view with portions fragmented of an alternative catheter assembly embodiment.

FIG. 5 is a cross-sectional view of a plurality of stacked multi-temperature devices.

### DETAILED DESCRIPTION OF THE PREFERRED EMBODIMENT

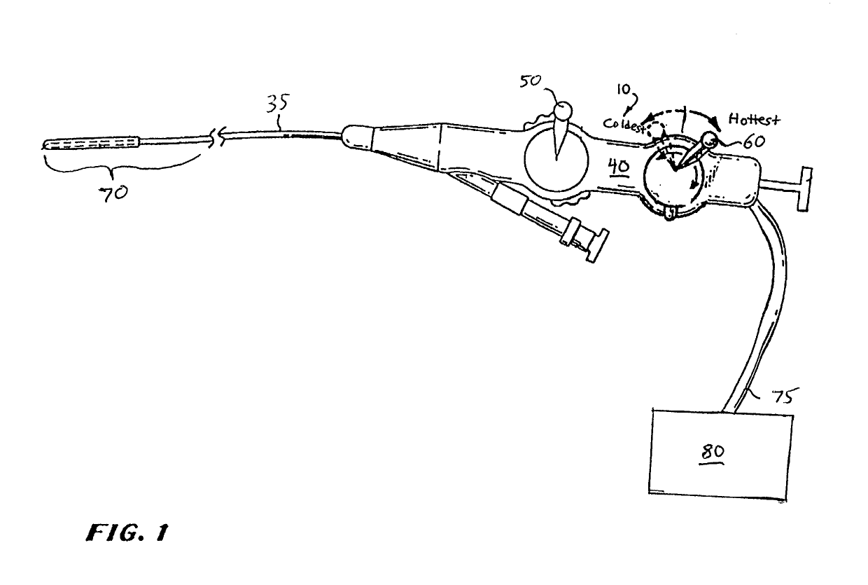

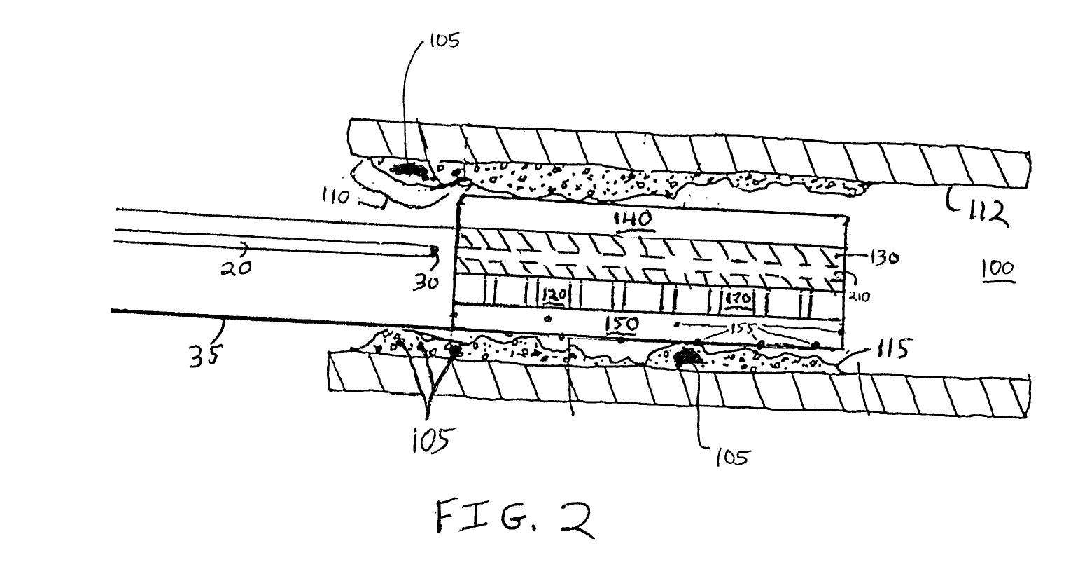

With respect to FIGS. 1 and 2, a plaque stabilizer device 10 includes a catheter 20 having a distal tip 30 and a guide sheath 35, and a handle 40 which can have a steering control 50 for the distal tip 30 of catheter 20 and a temperature control 60 for a catheter assembly 70. For example, moving the temperature control 60 toward the “hottest” position increases the temperature, while moving the temperature control 60 toward the “coldest” position decreases the temperature. FIG. 1 does not illustrate the catheter 20 or distal tip 30 because these structures are covered by the guide sheath 35. A cable 75 extending from the rear of handle 40 connects to a control box 80, which functions as a power supply and a temperature monitor for the catheter assembly 70. The catheter assembly 70 is attached to handle 40 via guide sheath 35, through which catheter 20 may freely pass.

With respect to FIG. 2, the catheter assembly 70 is depicted within a human artery 100 having unstable arterial plaque 105 within inflamed and unstable lipid pools 110 The material in these pools 110 can be congealed by application of heat and/or cold. This thickening or solidification will reduce the possibility of a sudden release of the dangerous plaque 105 from the lipid pool 110.

The lipid pool 110 is typically an entrapped volume between the interior vessel wall 112 and a layer of stable plaque 115 holding a pool of unstable plaque 105. Lipid pools 110 containing arterial plaque 105 that becomes inflamed and unstable incur an increased risk of ulceration and rupture. When lipid pools 110 rupture, a blood clot typically forms which will likely grow and cause a myocardial infarction.

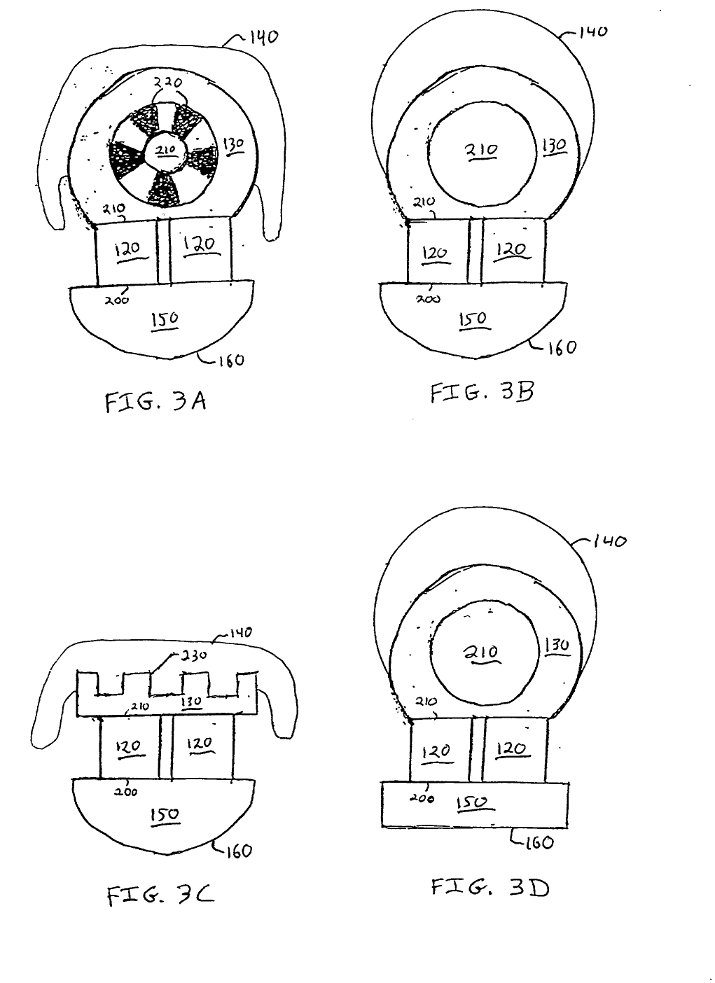

In one embodiment of the present invention, the catheter assembly 70 is to be used as a heating/cooling apparatus for transforming inflamed and unstable liquid lipid pools 110 into stable or solidified, non-flowing lipid pools 110, which are less likely to suffer a rupture. The catheter assembly 70 includes multi-temperature devices 120, heat exchanger 130, expander 140 and therapy plate 150 having temperature sensors 155. In all of the embodiments and figures, multi-temperature device 120 may be, for example, a device which has both a heating and a cooling surface (e.g., a heating-cooling device) or a Peltier device. Likewise, any reference to a Peltier device in the description of the embodiments can be changed to a different kind of multi-temperature device. Preferably, the multi-temperature device 120 is a Peltier device. As depicted in FIGS. 3A-3C, therapy plate 150 has a rounded tissue-contacting surface 160 to provide maximum surface area contact with internal artery wall 112. Alternatively, the tissue-contacting surface 160 can be flat as depicted in FIG. 3D, or other shapes as required or desired.

Importantly, lipid pools 110 containing inflamed and unstable plaque 105 have associated localized temperature variations of the interior blood vessel wall 112. Temperature sensors 155 are used to sense localized temperature variations of the blood vessel wall 112 within human artery 100. By detecting the presence of increased temperatures, the sensors 155 can anticipate the presence of lipid pools 110 containing inflamed and unstable plaque 105, which have up to a two and a half degree increase in temperature.

Preferably, the temperature sensors 155 comprises a plurality of thermistors 155 or thermocouples 155 located on the tissue-contacting surface 160 of the therapy plate 150 so that they can directly monitor tissue temperature. Additional temperature sensors 155 can be imbedded within the therapy plate 150 in order to more accurately monitor its temperature. The thermocouples 155 are electrically connected to the control box 80 by sensor leads (not shown), which extend through respective therapy plate through holes (not shown). Therapy plate 150 is preferably made of metal such as copper or silver, but can alternatively be made of any thermally conductive material.

Multi-temperature devices 120 are small solid-state devices that typically operate as heat pumps and in this example embodiment, are Peltier devices. When a DC current of one polarity is supplied by control box 80 in one direction, heat is moved from the bottom side 200 of the Peltier devices 120 to top side 210, where it must be removed with a heat sink. A “cold” bottom side 200 can be used to solidify or “freeze” inflamed and unstable lipid pools 110 located within artery 100. If the current from control box 80 is reversed, the polarity of the Peltier devices 120 switches and bottom side 200 becomes the “hot” side. A “hot” bottom side 200 can be used to solidify inflamed and unstable lipid pools 110 by “cooking” the affected areas. The current can then be reversed to cool the affected area to reduce any thermal damage to the vessel walls

With respect to FIG. 2, a layer of multi-temperature devices 120, such as Peltier devices, is in thermal contact with therapy plate 150, which is in direct contact with the interior surface of the vessel, which in this view is plaque 115. Using temperature control 60, a physician can adjust the electrical current supplied by control box 60 to power multi-temperature devices 120 and apply heat or cold to inflamed and unstable lipid pools through therapy plate 150. Additionally, control box 60 has a read out of arterial wall temperature and therapy plate 150 temperature to aid the physician in regulating the current supplied to the multi-temperature devices 120 and thereby the regulating the provision of heat/cold to the vessel.

In order to achieve a greater temperature differential, a plurality of multi-temperature devices 120 may be stacked on top of each other. The maximum difference in temperature of an individual device 120 is dependent on the magnitude of the electrical current and the temperature of the other side of the device 120. By stacking additional devices 120 on top of each other, the maximum difference in temperature can be increased until the electrical dissipation overloads the thermal capabilities of the multi-temperature device 120.

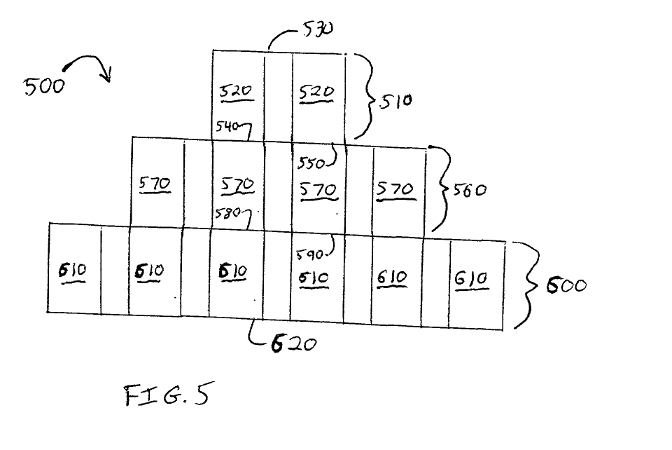

With respect to FIG. 5, in the operation of a three-layer stacked multi-temperature device 500, such as a Peltier device, for obtaining a cold surface, a first layer 510 has a number of devices 520 with “cold” side 530. The “hot” side 540 of these devices 520 abuts a “cold” side 550 of a second layer 560 having more devices 570 than the first layer 510 in order to account for the additional energy caused by the inefficiency of the first layer 510 of devices 520. The “hot” side 580 of the second layer 560 abuts “cold” side 590 of a third layer 600. The third layer 600 consequently needs to have even more devices 610 in order to remove the thermal energy from “hot” side 620. In other words, the devices 520 are stacked so the total temperature effect is cumulative.

If the multi-temperature device 500 is used as a cooling device, the “hot” thermal energy from “hot” side 620 must be removed or dissipated. Conversely, if the multi-temperature device 500 is used as a heating device, the polarity of the device 500 is switched such that one side 530 produces “hot” thermal energy and other side 620 produces “cold” thermal energy, which must be removed or dissipated. Additional thermal energy due to the inefficiency of the multi-temperature device 500 will also have to be removed. Both the thermal and additional energy due to the inefficiency of the multi-temperature device 500 are preferably dissipated by heatsinking using the heat exchanger 130.

With respect to FIGS. 2, 3B and 3D, heat exchanger 130 preferably is an elongated, thermally conductive cylinder having a circular aperture 210 for the circulation of blood and for the passage of catheter 20, but may be formed in other configurations. The aperture 210 could be, for example, an elliptical, oval, rectangular, or other shaped aperture. Also preferably, heat exchanger 130 is made of metal such as copper or silver, but can alternatively be made of any thermally conductive material. Heatsinking is best accomplished by using the blood circulating through aperture 210 of heat exchanger 130 to carry the undesired energy away. Alternatively, heatsinking or removing unwanted thermal energy may be achieved by circulating a cold media such as water or gas through the heat exchanger 130.

With respect to FIGS. 3A and 3C, the heat exchanger 130 can consist of several alternative configurations. Referring to FIG. 3A, a plurality of starburst fins 220 surround the internal perimeter of circular aperture 210. These fins 220 are integral with heat exchanger 130 and made with the same or different thermally conductive material such that the surface area for conducting the undesired thermal energy is significantly increased. FIG. 3C depicts an alternative embodiment for a heat exchanger 130, which has a serrated blood-contacting surface 230 having a plurality of recesses for increased surface area contact and enhanced heat conducted to the blood stream. Increased surface area allows more unwanted thermal energy to be transferred away from the therapy plate.

With respect to FIGS. 2 and 3A-3D, the catheter assembly 70 may include expander 140, if desired, for correctly positioning the therapy plate 150 against the plaque on the artery wall 112. The expander is preferably a balloon 140 that is capable of expansion from a first collapsed configuration as shown in FIGS. 3A and 3C to a second, expanded configuration as shown in FIGS. 2, 3B and 3D. Balloon 140 is elastic and inflatable with fluid such as saline, air, CO₂, or another fluid through inflation lumen (not shown) extending through guide sheath 35. In use, the catheter assembly 70 is inserted and advanced in the blood vessel 100 while in the collapsed configuration. At predetermined locations, the catheter assembly 70 is stopped and balloon 140 is inflated to the expanded configuration pressing therapy plate 150 against artery wall 112. Temperature sensors 155 detect areas of inflamed and unstable plaque 105 by detecting increased temperatures. Once these areas are identified, the catheter assembly 70 is used to stabilize the plaque by heating and/or cooling the affected areas.

The congealed or solidified plaque resultant from the procedure can either be left in place if stable or removed by procedures such as atherectomy. Atherectomy type catheters are used to remove material from the blood vessel walls and therefor remove the congealed or non-fluid material before it causes a blockage. These catheters have a rotating blade that cuts the material to be removed from the vessel wall and captures it inside a cylinder ahead of the rotating blade. Performing atherectomy on solidified plaque is far less dangerous than when the plaque is in liquid form because the possibility of toxic fluid leaking out, mixing with the blood stream and forming a blood clot has been significantly reduced.

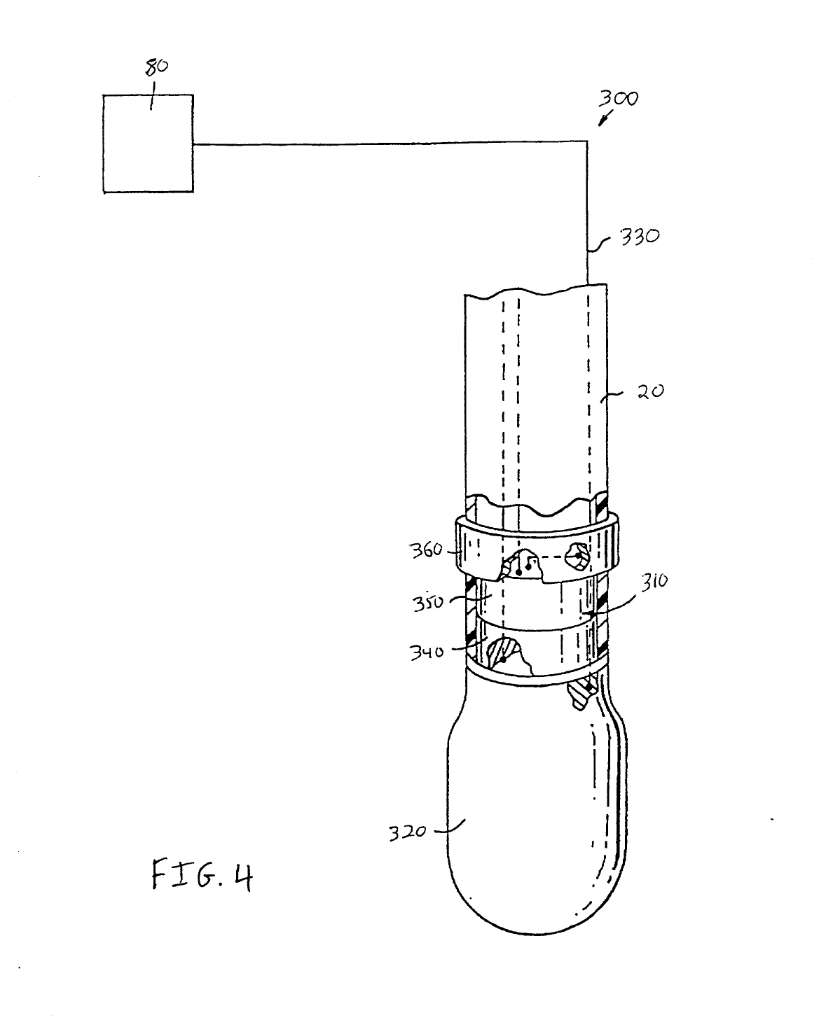

In the alternative embodiment shown in FIG. 4, a catheter assembly 300 includes a conventional Peltier diode 310 associated with an electrode 320 on the distal tip 30 of catheter 20, which is also electrically coupled by wire 330 to the control box 80. The materials of the diode 310 are preferably complex alloys, one doped “p” and the other doped “n”, creating a diode junction. An applied voltage potential passes current from a control box 80 through the junction. The polarity of the voltage creates a “cold” side 340 of the diode 310, which is coupled in thermal conductive contact to the electrode 320, and, a “hot” side 350 of the diode 310, which is coupled in thermal conductive contact to a heat exchanger 360. The heat exchanger 360 can be carried on the catheter 20 away from the electrode 320 and in contact with the blood pool.

The passage of current through the diode 310 creates a heat pump action from cold side 340 to hot side 350, conducting heat energy from the thermal mass of the electrode 320 to the heat exchanger 360. Heat energy can thus be transferred from the thermal mass of the electrode to cool it. Conversely, the polarity of the diode 310 can be reversed so that heat energy is transferred to the thermal mass of the therapy plate to heat it.

Any one or more of the features depicted in FIGS. 1-4, or described in the accompanying text, may be interchanged with that of another figure to form still other embodiments.

While preferred embodiments and methods have been shown and described, it will be apparent to one of ordinary skill in the art that numerous alterations may be made without departing from the spirit or scope of the invention. Therefore, the invention is not limited except in accordance with the following claims.

I claim:

1. A method of solidifying plaque on the interior of a blood vessel comprising the steps of:
inserting a catheter assembly having an expandable portion into the blood vessel while the expandable portion is in a collapsed configuration;
advancing the assembly in the blood vessel;
stopping at a plurality of predetermined locations along the blood vessel and expanding the expandable portion to an expanded configuration such that a plurality of temperature detectors contact the interior wall of the blood vessel;
sensing variation in blood vessel temperature at the plurality of predetermined locations along the interior wall of the blood vessel; and
applying thermal energy to, or removing thermal energy from, areas of increased temperature to reduce the possibility of leakage of the plaque into the blood vessel.

2. The method of claim 1 wherein the step of applying or removing thermal energy involves the use of at least one multi-temperature device having a hot side and a cold side and being powered by an electrical current having a polarity.

3. The method of claim 2 wherein the catheter assembly further includes a therapy plate positioned in between the at least one multi-temperature device and the interior of the blood vessel wall; the therapy plate having a tissue-contacting surface adapted to abut the interior of the blood vessel wall.

4. The method of claim 3 wherein the step of sensing blood vessel wall temperature variation further includes using temperature sensors located on the tissue-contacting surface of the therapy plate to sense variations in blood vessel tissue temperature.

5. The method of claim 4 further including the step of detecting the temperature of the therapy plate by using additional sensors imbedded within the therapy plate.

6. The method of claim 4 wherein the temperature sensors include thermocouples, thermistors or the like.

7. The method of claim 2 further including the step of reversing the polarity of the electrical current to change the hot side to the cold side and the cold side to the hot side.

8. The method of claim 2 further including the step of removing additional, undesired energy caused by the at least one multi-temperature device by using a heat exchanger as a heat sink.

9. The method of claim 8 wherein the step of removing undesired energy includes circulating fluid through an aperture in the heat exchanger.

10. The method of claim 2 wherein the at least one multi-temperature device includes a plurality of Peltier devices stacked one on top of another to increase the overall energy differential.

11. The method of claim 8 wherein the step of removing undesired energy includes using the blood circulating through an aperture in the heat exchanger to carry away the additional, undesired energy caused by the multi-temperature device.

12. The method of claim 8 wherein the heat exchanger includes an aperture lined with internal starburst fins.

13. The method of claim 8 wherein the thermal exchanger includes a serrated blood-contacting plate having a plurality of recesses.

14. The method of claim 1 further including the step of removing the solidified plaque resultant from the procedure.

15. The method of claim 14 wherein the solidified plaque is removed using atherectomy.

16. The method of claim 1 wherein the catheter assembly includes an expander, a heat exchanger, a therapy plate and at least one Peltier device.

17. The method of claim 16 wherein the expander is a balloon adapted to be inflated.

18. The method of claim 16 wherein the at least one Peltier device includes a plurality of Peltier devices stacked one on top of another to increase the overall energy differential.

19. The method of claim 16 further including a temperature control to monitor the temperature of the therapy plate.

20. The method of claim 16 further including a control box having a readout for artery wall temperature and therapy plate temperature.

21. The method of claim 2 wherein the multi-temperature device is a Peltier device.

22. An apparatus for solidifying plaque on the interior wall of a blood vessel comprising:
a catheter assembly for insertion into and advancement through a blood vessel, wherein the catheter assembly is to be stopped at predetermined intervals along the blood vessel; and
at least one multi-temperature device having a hot side and a cold side for solidifying plaque on the interior wall of the blood vessel.

23. The apparatus of claim 22 further including a plurality of temperature detectors for contacting the interior wall of the blood vessel and identifying areas of increased temperature indicative of inflamed and/or unstable plaque.

24. The apparatus of claim 22 wherein the catheter assembly is in a collapsed configuration during insertion and advancement through the blood vessel.

25. The apparatus of claim 24 wherein the catheter assembly is in an expanded configuration during solidification of the plaque.

26. The apparatus of claim 25 wherein the at least one multi-temperature device is powered by an electrical current having a polarity.

27. The apparatus of claim 22 wherein the catheter assembly includes a therapy plate positioned in between the at least one multi-temperature device and the interior of the blood vessel wall; the therapy plate having a tissue-contacting surface adapted to abut the interior of the vessel wall.

28. The apparatus of claim 27 further including temperature sensors located on the tissue-contacting surface of the therapy plate for sensing variations in blood vessel tissue temperature.

29. The apparatus of claim 28 further including additional sensors imbedded within the therapy plate for detecting the temperature of the therapy plate.

30. The apparatus of claim 28 wherein the temperature sensors are thermocouples or thermistors.

31. The apparatus of claim 26 wherein the polarity of the electrical current can be reversed to change the hot side to the cold side and/or vice-versa.

32. The apparatus of claim 22 wherein the catheter assembly includes a heat exchanger for removing additional, undesired energy caused by the at least one multi-temperature device.

33. The apparatus of claim 32 wherein the heat exchanger has an aperture extending the length of the heat exchanger for the passage of blood.

34. The apparatus of claim 33 wherein blood is used to carry away the additional, undesired energy caused by the at least one multi-temperature device.

35. The apparatus of claim 33 wherein the aperture is lined with internal starburst fins.

36. The apparatus of claim 32 wherein the heat exchanger includes a serrated blood-contacting plate having a plurality of recesses.

37. The apparatus of claim 22 wherein the solidified plaque resultant from the procedure is to be removed using atherectomy.

38. The apparatus of claim 22 wherein the catheter assembly includes an expander, a heat exchanger, a therapy plate and at least one multi-temperature device.

39. The apparatus of claim 38 wherein the expander is an elastic balloon adapted to be inflated with a fluid.

40. The apparatus of claim 38 wherein the at least one multi-temperature device includes a plurality of multi-temperature devices stacked one on top of another to increase the overall energy differential.

41. The apparatus of claim 38 further including a temperature control to monitor the temperature of the therapy plate.

42. The apparatus of claim 38 further including a control box having a readout for artery wall temperature and therapy plate temperature.

43. The apparatus of claim 22 wherein the catheter assembly includes a catheter having an electrode on its distal tip.

44. The apparatus of claim 43 wherein the at least one multi-temperature device comprises a Peltier diode for cooling and heating the electrode.

45. The apparatus of claim 44 further including a heat exchanger.

46. The apparatus of claim 22 wherein the multi-temperature device includes a Peltier device.

47. The apparatus of claim 40 wherein the multi-temperature devices include Peltier devices.

48. A method of solidifying plaque on the interior of a blood vessel comprising the steps of:
inserting a catheter assembly into the blood vessel;
advancing the assembly in the blood vessel;
stopping at a plurality of predetermined locations along the blood vessel such contact the interior wall of the blood vessel;
sensing variation in blood vessel temperature at the plurality of predetermined locations along the interior wall of the blood vessel using a plurality of temperature detectors; and
applying thermal energy to, or removing thermal energy from, areas of increased temperature to reduce the possibility of leakage of the plaque into the blood vessel.

49. The method of claim 48 wherein the step of applying or removing thermal energy involves the use of at least one Peltier device having a hot side and a cold side and being powered by an electrical current having a polarity.

50. The method of claim 49 wherein the catheter assembly further includes a therapy plate positioned in between the at least one Peltier device and the interior of the blood vessel wall;
the therapy plate having a tissue-contacting surface adapted to abut the interior of the blood vessel wall.

51. The method of claim 50 wherein the step of sensing blood vessel temperature variation further includes using temperature sensors located on the tissue-contacting surface of the therapy plate to sense variations in blood vessel tissue temperature.

52. The method of claim 51 further including the step of detecting the temperature of the therapy plate by using additional sensors imbedded within the therapy plate.

53. The method of claim 51 wherein the temperature sensors include thermocouples or thermistors.

54. The method of claim 49 further including the step of reversing the polarity of the electrical current to change the hot side to the cold side and the cold side to the hot side.

55. The method of claim 49 further including the step of removing additional, undesired energy caused by the at least one Peltier device by using a heat exchanger as a heat sink.

56. The method of claim 55 wherein the step of removing undesired energy includes circulating fluid through an aperture in the heat exchanger.

57. The method of claim 49 wherein the at least one Peltier device includes a plurality of Peltier devices stacked one on top of another to increase the overall energy differential.

58. The method of claim 55 wherein the step of removing undesired energy includes using the blood circulating through an aperture in the heat exchanger to carry away the additional, undesired energy caused by the Peltier device.

59. The method of claim 49 wherein the heat exchanger includes an aperture lined with internal starburst fins.

60. The method of claim 49 wherein the thermal exchanger includes a serrated blood-contacting plate having a plurality of recesses.

61. The method of claim 48 further including the step of removing the solidified plaque resultant from the procedure.

62. The method of claim 61 wherein the solidified plaque is removed using atherectomy.

63. The method of claim 48 wherein the catheter assembly includes an expander, a heat exchanger, a therapy plate and at least one Peltier device.

64. The method of claim 63 wherein the expander is a balloon adapted to be inflated.

65. The method of claim 63 wherein the at least one Peltier device includes a plurality of Peltier devices stacked one on top of another to increase the overall thermal energy differential.

66. The method of claim 63 further including a temperature control to adjust and monitor the temperature of the therapy plate and vessel temperature.

67. The method of claim 63 further including a control box having a readout for artery wall temperature and therapy plate temperature.

68. An apparatus for solidifying plaque on the interior wall of a blood vessel comprising:
a catheter assembly having a collapsed configuration and an expanded configuration, the catheter assembly being in the collapsed configuration during insertion into and advancement through a blood vessel, the catheter assembly being in the expanded configuration during solidification of the plaque;
at least one Peltier device having a hot side and a cold side for solidifying plaque on the interior wall of the blood vessel, the at least one Peltier device being powered by an electrical current having a polarity wherein the polarity of the electrical current can be reversed to change the hot side to the cold side and/or vice-versa;
a plurality of temperature detectors adapted to contact the interior wall of the blood vessel to identify areas of increased temperature indicative of inflamed and/or unstable plaque;
a heat exchanger which removes undesired energy generated by the at least one Peltier device; and
a therapy plate positioned in between the at least one Peltier device and the interior of the blood vessel wall, the therapy plate having a tissue-contacting surface adapted to contact the interior of the vessel wall.

69. The apparatus of claim 68 further comprising at least one temperature sensor located on the tissue-contacting surface of the therapy plate for sensing a variation in blood vessel tissue temperature.

70. The apparatus of claim 69 further including an additional sensor embedded in the therapy plate for detecting the temperature of the therapy plate.

71. The apparatus of claim 69 wherein the temperature sensor is a thermocouple or thermistor.

72. The apparatus of claim 68 wherein the heat exchanger has an aperture extending the length of the heat exchanger for the passage of blood.

73. The apparatus of claim 72 wherein blood carries away the additional energy generated by the at least one Peltier device.

74. The apparatus of claim 72 wherein the aperture is lined with internal starburst fins.

75. The apparatus of claim 72 wherein the heat exchanger includes a serrated blood-contacting plate having a plurality of recesses.

76. The apparatus of claim 68 wherein the catheter assembly includes an elastic balloon adapted to be inflated with a fluid.

77. The apparatus of claim 68 wherein the at least one Peltier device includes a plurality of Peltier devices stacked one on top of another to increase the overall energy differential.

78. The apparatus of claim 68 further including a temperature control to monitor the temperature of the therapy plate.

79. The apparatus of claim 68 further including a control box having a digital readout for blood vessel wall temperature and therapy plate temperature.

80. The apparatus of claim 68 wherein the catheter assembly includes a catheter having an electrode on its distal tip.
